(19) United States
(12) Patent Application Publication
(10) Pub. No.: US 2017/0042565 A1
(43) Pub. Date: Feb. 16, 2017

(54) CANNULA GUIDED SURGICAL TOOL

(71) Applicant: Summit Medical Products, Inc., Sandy, UT (US)

(72) Inventors: Daniel D. B. Ellsworth, Little Elm, TX (US); Peter D. Hebbard, Milawa (AU); Gregg D. Niven, Kaysville, UT (US)

(73) Assignee: SUMMIT MEDICAL PRODUCTS, INC., Sandy, UT (US)

(21) Appl. No.: 14/823,623

(22) Filed: Aug. 11, 2015

(51) Int. Cl. A61B 17/32; A61B 17/3201

(52) U.S. Cl. CPC A61B 17/320036; A61B 17/3201; A61B 17/320016

(57) ABSTRACT

A surgical tool system includes a cannula assembly and a handle assembly. The cannula assembly includes a cannula having a sidewall about a central axis. The handle assembly includes a handle and a blade tool aligned with the central axis of the cannula. The blade tool has a blade edge and a back edge. The blade tool is longitudinally extendible relative to the handle. The blade tool is disposed within the cannula. In a deployed mode the back edge of the blade tool impinges on an interior surface of the sidewall of the cannula to force the blade edge of the blade tool to protrude laterally from the central axis of the cannula. This exposes the blade edge of the blade tool beyond an outer diameter of the sidewall of the cannula.

### BACKGROUND

The quest for practitioners of surgery is for more minimally invasive procedures for several reasons. The reasons range from quicker recovery for the patient, suture-less wounds, less pain, lower cost of the procedure, and in some cases improved recovery and patient satisfaction. One of the more common procedures that has surfaced where these desires are present is treating carpal tunnel syndrome.

The main intent of minimally invasive procedures used to treat carpal tunnel is to release pressure on the median nerve with minimal impact to surrounding tissue and with faster recovery times. The pressure may be due to swelling or injury due to repetitive motion of the hand and wrist. The pressure in carpal tunnel may be released by incising the tissue to reveal and incise the transverse carpal ligament along and parallel to the median nerve axis. The resulting freedom gained by incising the tendon is a release in pressure on the median nerve, removing the pain. Other areas of minimally invasive procedures that are being recognized include Plantar fasciotomy release, forearm or tendon sheath release dealing with trigger finger or trigger thumb. Other percutaneous surgical procedures are being tested and reviewed to determine risk versus value for these procedures and to evaluate patient comfort and results. As these procedures emerge, specialized tools may be used to allow the surgeon to treat these areas with minimal impact and with shorter healing times.

Most conventional release procedures use direct visualization with an endoscope or arthroscope to view the anatomy of the area treatment site prior to and during the procedure. Other procedures require cutting and opening and retraction of the treatment site to ensure visualization is direct and open. There are several disadvantages of these conventional approaches, including affecting a larger area on the patient, damaging surrounding musculature, and the additional trauma due to inserting various devices (e.g., endoscope) into the area for treatment for visualization and the increased duration of time for the healing process.

Some conventional tools exist to reduce healing time and improve patient results and satisfaction. One recent study compared open treatment methods with ultrasound guided methods for treatment of carpal tunnel syndrome. The net result was that ultrasound guided procedures are found to reduce healing time and to increase patient satisfaction and strength results following the procedure. Additionally with these developments in research, several tools have been developed over the years to improve the dissemination of fluids and to improve the placement of needles into tissues of interest along with minimally invasive tools and procedures.

Some methods use tools which are inserted into minimal percutaneous apertures. One of the tools includes a guide and support and a single blade for use in carpal tunnel surgery. Other devices include a specialized knife and specially fitted guide to assist in inserting the knife and guiding the knife throughout the cut. Unless the tissue is cut to reveal the anatomy being treated, an endoscope is normally used along with the device in order to assure the proper anatomy is severed. With this type of visualization, a larger incision is required to receive the appliance as well as the visualizing medium.

Another device includes an appliance with several sleeved components, with an inner cannula that is sheathed with a cannula with integral cutting blade and an outer sheath that protects the assembly and surrounding tissue during the insertion process into the volar aspect of the forearm. This device also requires the use of an endoscope for direct observation. This again requires the use of a larger diameter cannula assembly along with an additional visualization device.

Another device is designed for use with fascia cutting for relieving nerve pressure. The device contains a hinged blade housed within the outer diameter of a metal cannula. The device is inserted into the fascia via its cannula shaped tip. The tool is activated and the hinged blade is raised from within the outer diameter of the cannula. Cutting of the fascia is accomplished by the removal of the tool from the percutaneous aperture created by the cannula. Observation in this case is performed via an endoscope to ensure cutting of the proper anatomy has been accomplished.

### SUMMARY

Embodiments of a surgical tool system are described. In one embodiment, the surgical tool system is guided through a needle cannula. An embodiment of the surgical tool system includes a cannula assembly and a handle assembly. The cannula assembly includes a cannula having a sidewall about a central axis. The handle assembly includes a handle and a blade tool aligned with the central axis of the cannula. The blade tool has a blade edge and a back edge. The blade tool is longitudinally extendible relative to the handle. The blade tool is disposed within the cannula. In a deployed mode the back edge of the blade tool impinges on an interior surface of the sidewall of the cannula to force the blade edge of the blade tool to protrude laterally from the central axis of the cannula. This exposes the blade edge of the blade tool beyond an outer diameter of the sidewall of the cannula. Other embodiments of the surgical tool system are also described.

In another embodiment, a surgical apparatus includes a handle, a needle cannula, a surgical tool, and an actuator. The handle defines an interior cavity. The needle cannula is coupled to the handle. A longitudinal axis of the needle cannula is aligned with the interior cavity of the handle. The surgical tool extends from the interior cavity of the handle to a distal end of the needle cannula. The actuator includes an extension interface coupled to the surgical tool to move the surgical tool longitudinally within the needle cannula and, in a deployed mode, extend the surgical tool out of the distal end of the needle cannula. Other embodiments of the surgical apparatus are also described.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 illustrates one embodiment of a cannula guided surgical tool.

FIG. 2 illustrates a more detailed view of an indicator on a handle of the cannula guided surgical tool of FIG. 1.

FIG. 3 illustrates another embodiment of the cannula guided surgical tool of FIG. 1 with a blade tool deployed.

FIG. 4 illustrates a more detailed side view of the blade tool of FIG. 3.

FIG. 5 illustrates another view of the cannula guided surgical tool of FIG. 1 with the cannula detached from the handle and the blade tool.

FIG. 6 illustrates a sectional side view of the cannula guided surgical tool of FIG. 1.

FIG. 7 illustrates a sectional side view of the disassembled cannula guided surgical tool of FIG. 5.

FIG. 8 illustrates a more detailed view of the cannula member of the cannula guided surgical tool of FIG. 1.

FIG. 9 illustrates another embodiment of a handle and tool component for use with a cannula guided surgical tool.

FIG. 10 illustrates another embodiment of a handle and tool component and a cannula component for use with a cannula guided surgical tool.

FIG. 11 illustrates another embodiment of a cannula guided surgical tool having a scissor handle assembly.

FIG. 12 illustrates another embodiment of a cannula guided surgical tool having a tool port on a sidewall of the cannula.

FIG. 13 illustrates a more detailed view of the ported cannula guided surgical tool of FIG. 12.

FIG. 14 illustrates a more detailed view of the ported cannula guided surgical tool of FIG. 12 with the surgical tool extending from the sidewall port.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the described embodiments implement a surgical tool that is guided through a needle cannula. In a specific embodiment, a simple fabricated device combines the small diameter of a single cannula that has an enclosed blade housed within. This combination of a penetrating needle along with a deployable blade may be beneficial for use in minimally invasive surgical intervention.

In some embodiments the cannula may be any size, but certain embodiments utilize a needle of 14 gauge to 16 gauge. These sizes of cannula facilitate percutaneous insertion to the desired location of the procedure and allow the blade to be deployed to protrude from the central axis of the cannula and to cut fascia, ligaments, or other soft tissue anatomy. In some embodiments, the device may be used in carpal tunnel syndrome procedures, but other embodiments may be used in other procedures wherein a simple, self-contained device can be used. In other embodiments, other sizes of needles may be used. For example, some embodiments may use smaller needles such as 18 gauge and 20 gauge needles for maxi-facial or other dermatology/plastics procedures. Ultimately, any size of needle or cannula may be used so long as the tool to be deployed fits within the needle or cannula.

In further embodiments, the procedure may be performed under the observation of an ultrasound visualizing system.

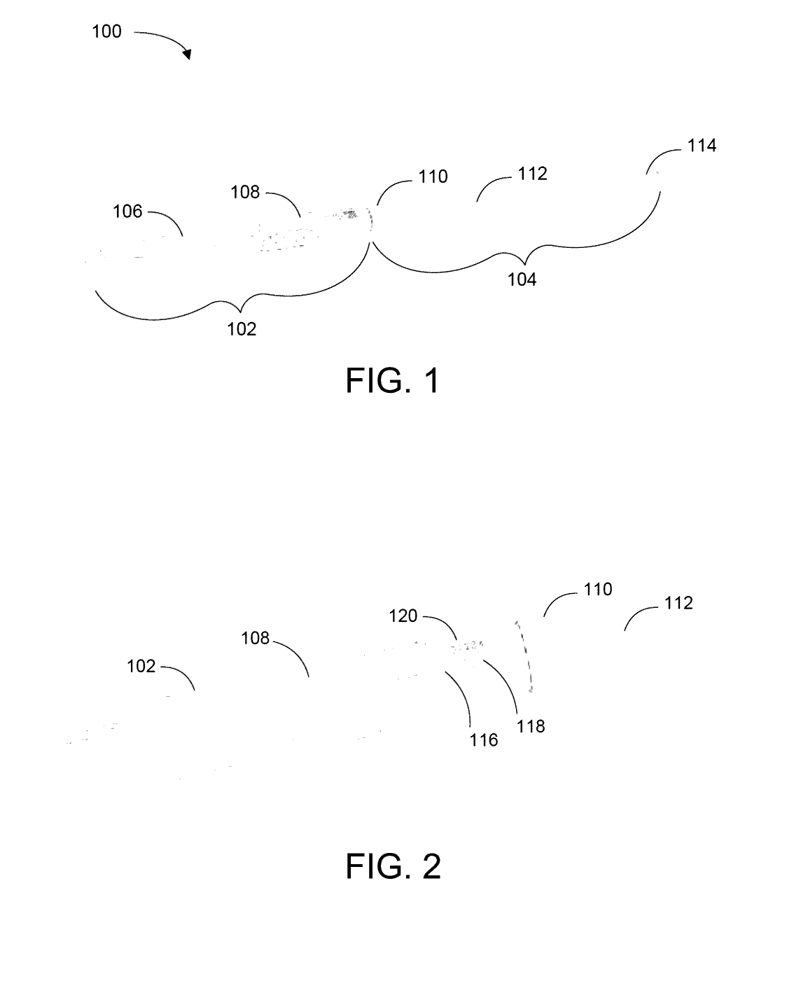

FIG. 1 illustrates one embodiment of a cannula guided surgical tool system 100. The illustrated surgical tool system 100 includes several component parts. However, other embodiments of the surgical tool system 100 may omit some of the illustrated component parts without detracting from aspects of functionality that distinguish it from conventional surgical tools. Alternatively, other embodiments of the surgical tool system 100 may include equivalent component parts to perform the same or similar functionality. Other embodiments of the surgical tool system 100 may include additional component parts to accomplish functionality that is complementary to the functionality described herein.

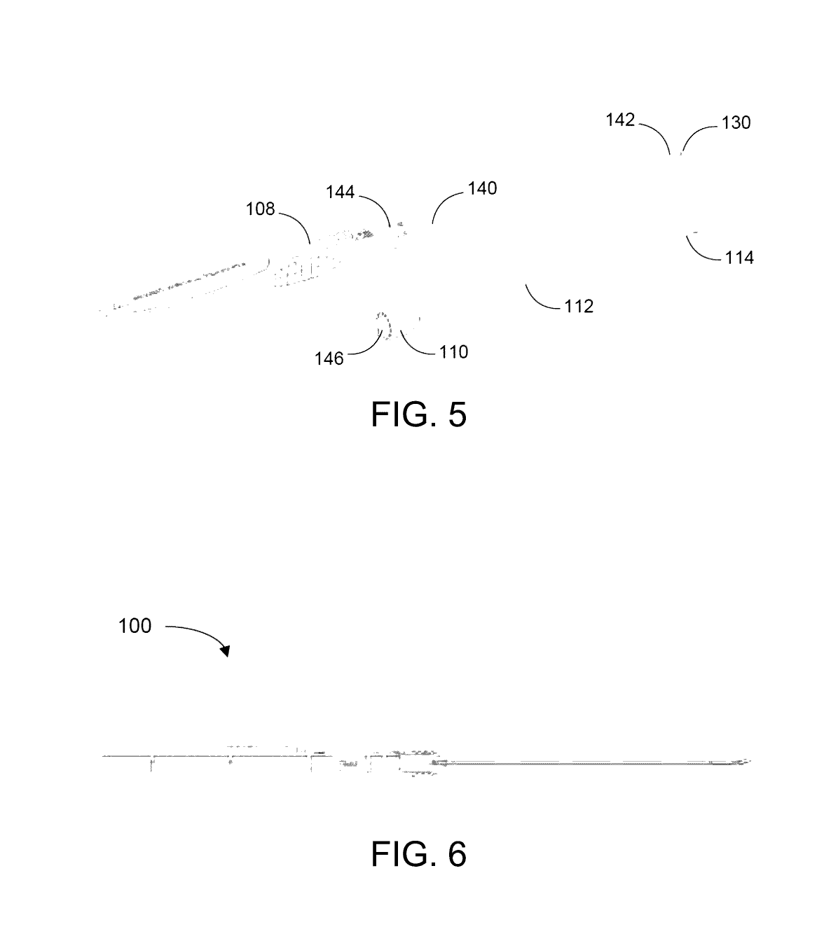

The depicted surgical tool system 100 incorporates two basic assemblies, including a handle assembly 102 and a cannula assembly 104. The handle assembly 102 includes a grip 106 and an actuator 108. Although not shown in FIG. 1, the handle assembly 102 also includes a surgical tool (see FIG. 5). The surgical tool is located within an interior cavity of the grip 106 and extends through the cannula assembly 104. The surgical tool is connected to the actuator 108, which moves the surgical tool within the cannula assembly 104.

The cannula assembly 104 includes a coupling 110 and a needle (i.e., cannula) 112. The coupling 110 helps attach the cannula assembly 104 to the handle assembly 102. A proximal end of the needle 112 is aligned with the internal cavity of the handle 102 in order to allow the surgical tool to pass through under the control of the actuator 108.

In general, the surgical tool system 100 allows a user to insert a distal end 114 of the needle 112. The user can then control deployment and retrieval of the surgical tool out of the distal end 114 of the needle 112. A variety of surgical tools may be implemented with the surgical tool system 100.

Various materials may be used for the construction of the surgical tool system 100. As examples, embodiments of the surgical tool system 100 may be injection molded plastic or machined metal and plastic components. In one embodiment, the surgical tool system 100 includes molded plastic components. However, other materials could be used if a more permanent or reusable device was desired. Material construction choices may be any material that best fits the needs to users of the tools and are familiar with the art.

In some embodiments, the handle assembly 102 and the cannula assembly 104 are joined together via a cap or other coupling 110 which holds a cannula adapter (see FIG. 5) to the distal end of the handle assembly 102. The connection may be made using a mating thread union or any other suitable connector.

FIG. 2 illustrates a more detailed view of an indicator 120 on the handle assembly 102 of the surgical tool system 100 of FIG. 1. In particular, the actuator 108 of the handle assembly 102 is configured to move in order to move the position of the attached surgical tool. In the illustrated embodiment, the actuator 108 is a slider, which easily facilitates sliding the surgical through the needle 112 of the cannula assembly 104. However, other types of actuators may be implemented, in which case the movement of the actuator 108 may be linear, rotational, or any other mode of movement.

The depicted actuator 108 has an attached position indicator 116. As the actuator 108 moves, the position indicator 116 also moves relative to a series of indication marks 118 and/or designations 120. The indication marks 118 and designations 120 provide the user with an understanding of the distance that the surgical tool extends out of the distal end 114 of the needle 112 when the needle is inserted into a patient and is not viewable.

The movement of the actuator 108 may be smooth and continuous or, in other embodiments, may be stepped at discrete intervals. In either case, the position indicator 116, indication marks 118, and indication designations 120 help convey the distance that the surgical tool moves. The indications 118 and designations 120 may correlate to standardized measurement units (e.g., 0-4 millimeters) of longitudinal movement of the surgical tool. Alternatively, they may indicate lateral movement of the end of the surgical tool away from the adjacent perimeter of the needle 112. Other embodiments may utilize other standards of measurement or, in some case, may provide indications that do not correlate directly to a standard measurement unit in a particular direction. Additionally, although the illustrated embodiment includes 5 numbered positions, other embodiments may include a different number of positions, different position step sizes, or other graphical indications instead of numbers.

As illustrated, the actuator 108 may be partially or fully recessed within a depression in the shell of the housing assembly 102. For example, the actuator 108 may be disposed within a recessed feature on the top of the handle assembly 102. Depending on the nature of the actuator 108, the actuator 108 may have contours or other surface features to fit the thumb or finger of a user to grip and move the actuator 108 to a specific position within the range of positions. The surface features may facilitate movement in multiple directions, for example to extend or retrieve the surgical tool.

In an alternative embodiment, the actuator 108 may include a male projection on the bottom. The male projection may engage with one or more recesses corresponding to locking positions of the actuator. In one example, the recesses are spaced apart at one millimeter intervals. Engagement of the actuator with an engagement point may provide tactile and/or audible feedback to the operator during the positioning of the surgical tool.

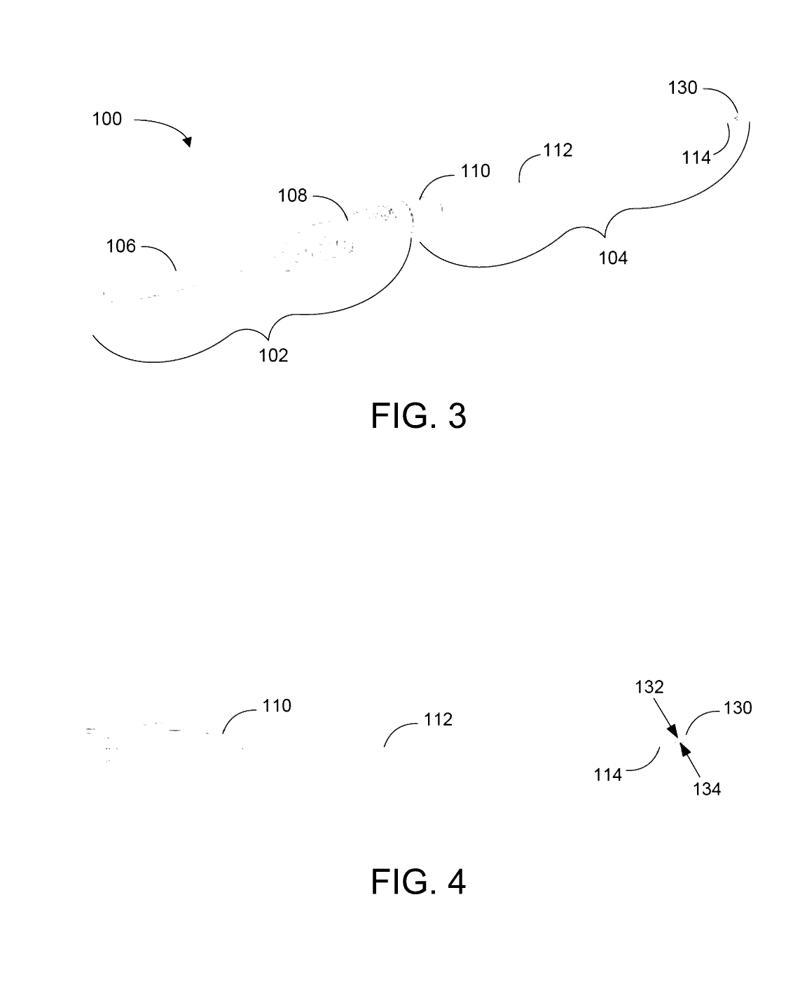

FIG. 3 illustrates another embodiment of the cannula guided surgical tool system 100 of FIG. 1 with a blade tool 130 deployed from the distal end 114 of the needle 112. In a storage mode (or non-deployed mode), the blade tool 130 is retrieved within the needle 112 so that it is not in contact with the soft tissues of a patient when the needle 112 is inserted. After insertion of the needle 112, the operator can use the actuator 108 to extend the blade tool 130 out of the distal end 114 of the needle 112 and perform any desired surgical procedures. Any position in which the blade tool 130 extends out of the needle 112 can be considered a deployed position. Once the procedure is complete, the operator can retrieve the blade tool 108 to position it back within the needle 112, then extract the needle 112 without unnecessary cutting to the surrounding soft tissues.

FIG. 4 illustrates a more detailed side view of the blade tool 130 of FIG. 3. In the depicted embodiment, the blade tool 130 includes a blade edge 132 and a back edge 134. The blade edge 132 is sharp for cutting tissues, while the back edge 134 is not sharp. In some embodiments, the back edge 134 is used to push the blade edge 132 out of the needle 112 when the actuator 108 moves to the deployed position and the back edge 134 makes contact with an interior surface of the distal end 114 of the needle 112. For example, a touhy cannula has a curved distal end 114 which will position an interior surface to receive contact from the back edge 134 of the blade tool 130 and force the blade edge 132 (or a larger portion of the blade tool) to extend out of the cannula opening.

Once extended or deployed by the actuator 108, the blade tool 130 may be locked into a deployed position for use as a surgical knife to allow cutting as the surgical tool system 100 as a whole is extracted from the patient. The cutting may be applied to tendons or fascia as needed by the clinician.

Although a specific example of a blade tool 130 is included, other embodiments of the surgical tool system 100 may include different surgical tools. For example, other tools not shown may include tools for removing and capturing biopsy samples, grasping to pull or move tissue for more access during a procedure, or for cutting using a scissor cutting tool. The types of tools which may be implemented in a particular embodiment of the surgical tool system 100 are not limited, as long as the surgical tool can fit within the inside diameter of the needle cannula 112.

Also, although the illustrated embodiment shows a needle 112 with a curved distal end 114, other embodiments may utilize a straight needle or another type of formed needle. In some instances, the shape of the tip 114 of the needle 112 may be chosen or designed for a specific type of insertion or access to a particular surgical site (e.g., a site requiring special shapes or angles to obtain a preferred position).

FIG. 5 illustrates another view of the cannula guided surgical tool system 100 of FIG. 1 with the cannula assembly 104 detached from the handle 102 and the blade tool 130. In this disassembled view, the blade tool 130 is coupled via an extension 140 and joint 142 and an internal connector (not visible) to the actuator 108.

In some embodiments, the internal connector is a stainless steel sleeve that is crimped to the extension 140 and driven by the actuator 108. The sleeve allows accurate positioning of the blade 130 relative to the handle 102 and the distal end 114 of the needle 112. Accurate positioning of the blade extension 140 and retraction not only protects the blade 130 during insertion and deployment, but also provides accurate repeatable feedback for the clinician to understand the true position of the end of the blade 130 so future results or uses of the tool are more predictable. The control and retention of the blade 130 and extension 140 via the sleeve, allows the blade 130 to remain in position, secured to the handle 102, and be totally separated from within the needle cannula 112 while leaving the cannula 112 inserted into the surgical site.

The extension 140 provides rigidity to the surgical tool so that movement of the actuator 108 translates into corresponding movement of the blade 130. The joint 142 allows the blade 130 to move out-of-axis relative to the extension 140. In some embodiments, this lateral movement facilitates lateral exposure of the blade edge 132 of the blade 130 from the distal end 114 of the needle 112.

In some embodiments, the blade 130 is a small diameter wire formed to have a blade edge 132. For example, the blade 130 may be formed from a 0.045″ diameter wire on the proximal end with a ground blade edge 132. Other embodiments may use other sizes of wire or other methods of forming the blade edge 132.

Similarly, the joint 142 is also formed from the same wire or material used for the extension 140 and the blade 130. In some embodiments, the joint 142 is formed as a flexible web on the distal end of the extension 140. The blade 130 and joint 142 may be cut and ground and maintain their contours within the outside diameter of the wire from which they are formed, so as to maintain their ability to fit within the needle cannula 112.

In some embodiments, the use of a long flexible web for the joint 142 has certain advantages, including the ability of the wire supporting the blade to flex repeatedly with less stress concentration and subsequent fatigue than if the joint 142 was shorter in length. A shorter web design concentrates stresses of the metal, thereby enhancing the possibility of material fatigue and possible separation. Preventing separation of the blade 130 from the supporting extension 140 avoids a safety hazard that might otherwise require surgical intervention for the retrieval of the blade 130 or with other surgical grasping means.

In some embodiments, the length of the blade 130 may be approximately 3-4 millimeters in length, and the flexible web forming the joint 142 and connecting the blade 130 to the wire extension 140 may be approximately 6-10 millimeters in length. The lengths of these features may be a function of the internal bore dimensions of the cannula 112. If the cannula 112 is formed as a touhy cannula with a radius in the distal end 114, then the radius of the touhy bend of the cannula has a direct relationship to the length of the blade 130 and joint 142. The larger bend radius of the cannula 112, the longer the blade 130. In some embodiments, the cannula bend radius compared to blade length is approximately a 3.5:4 ratio. Other embodiments may have other dimensional relationships compatible with the type of tool and the type and/or size of the cannula.

At the point of entry of the extension 140 into the internal cavity of the handle 102, the handle 102 includes a connector or coupling 144. This connector or coupling 144 is compatible with the corresponding connector or coupling 110 of the cannula assembly 104. The cannula assembly 104 also includes an adapter 146 which mates to a corresponding adapter (not shown) within the distal end of the handle 102. The adapter within the handle has an opening small enough to hold the needle with a pass through aperture through the center of the adapter. Both apertures are connected concentrically via the adapters, which can allow a clear path through the adapter to be used for insertion of other devices or the delivery of fluids from the proximal luer end to the distal end of the cannula.

The luer tapered end of the adapter may be sized using industry standard defined fitting diameters and angles to allow it to be attached to commonly used medical tubing and fitting sets used in infusion therapy or intravenous therapies commonly used in the medical industry. Small tabs on the adapter also enable the locking of the medical industry fittings to the adapter, in order to be able to present a more secure fluid path also enabling higher fluid pressures to be used should that be desirable. One of the tabs on the adapter may be a keying tab that forces the correct alignment and orientation of the cannula and fitting when the cannula is installed in the handle. The cannula assembly can be attached to the handle assembly via the connector or coupling 110 which is threaded on the inside and mates to the corresponding threaded distal end 144 of the handle assembly 102. Other embodiments may use other types of adapters, connectors, or couplings.

In some embodiments, the handle assembly 102 is a handle casting sized in length to permit it being held and manipulated by a right or left handed clinician. Also, the handle casting may be sized in diameter to allow comfortable grasping and holding to permit pressures great enough to help the needle to penetrate the patient tissue. The size and geometry of the handle casting also allows it to be grasped firmly enough to be pressed into or withdrawn to gain access percutaneous to the surgical site. As described above, a portion of the handle is hollowed out in the center to form the internal cavity. In some embodiments, the bottom of the handle also may be hollowed out or open to allow the surgical tool to be inserted and retained.

The needle 112 of the cannula assembly 104 may be fabricated from any suitable material such as 304 or 316 medical grade stainless steel. The hardness of the medical grade steel allows the distal end 114 of the needle 112 to be sharpened and to maintain a surgical edge during use. Other embodiments may be fabricated from other bio compatible materials.

In one embodiment, the cannula assembly 104 uses a 16 gauge stainless steel cannula 112 for blade containment and control. In other embodiments, the diameter of the cannula 112 may range from 16 gauge to 13 gauge to be fabricated from needle stock, or if the use of the tool permits access or the use of specialized surgical tools too small to fit within the diameter of needle cannula 112. Other embodiments may use other sizes of cannula, depending on the type of surgical tool to be used or the type of surgical procedure to be performed, or the accessibility of the surgical site.

In some embodiments, the outer surface of the cannula 112 may be treated with a surface treatment, chemical etch, mechanical treatment, or coating for visualization of the needle beneath the skin. Other embodiments may remain untreated or uncoated. Certain surface treatments may improve one or more methods of visibility by, for example, creating a more reflective surface (where reflectivity of light or non-visible wavelengths of energy can be detected) or a more visible surface where direct visualization is used. Coatings may improve surface reflectivity by altering the reflection coefficient, by changing one or more indices of refraction, by altering the amplification of the reflected signal (through constructive or destructive energy), by impacting the specular or diffuse nature of the reflected signals, by changing directionality of the reflected signals, or through another approach which alters the quantity, quality, or directionality of the reflected signals. This functionality may be helpful for catheter or needle placement, enabling this cannula 112 to be viewed using a visualization system (not shown) such as a medical ultrasound system, a fluoroscope or x-ray system, or another visualization system. Alternatively, the surface of the cannula 112 may be untreated, and the visualization may be performed directly using an endoscope or arthroscope.

FIG. 6 illustrates a sectional side view of the cannula guided surgical tool system of FIG. 1. In this view, an embodiment of the molded structure of the handle assembly 102 is visible, including several open cavities along the bottom surface.

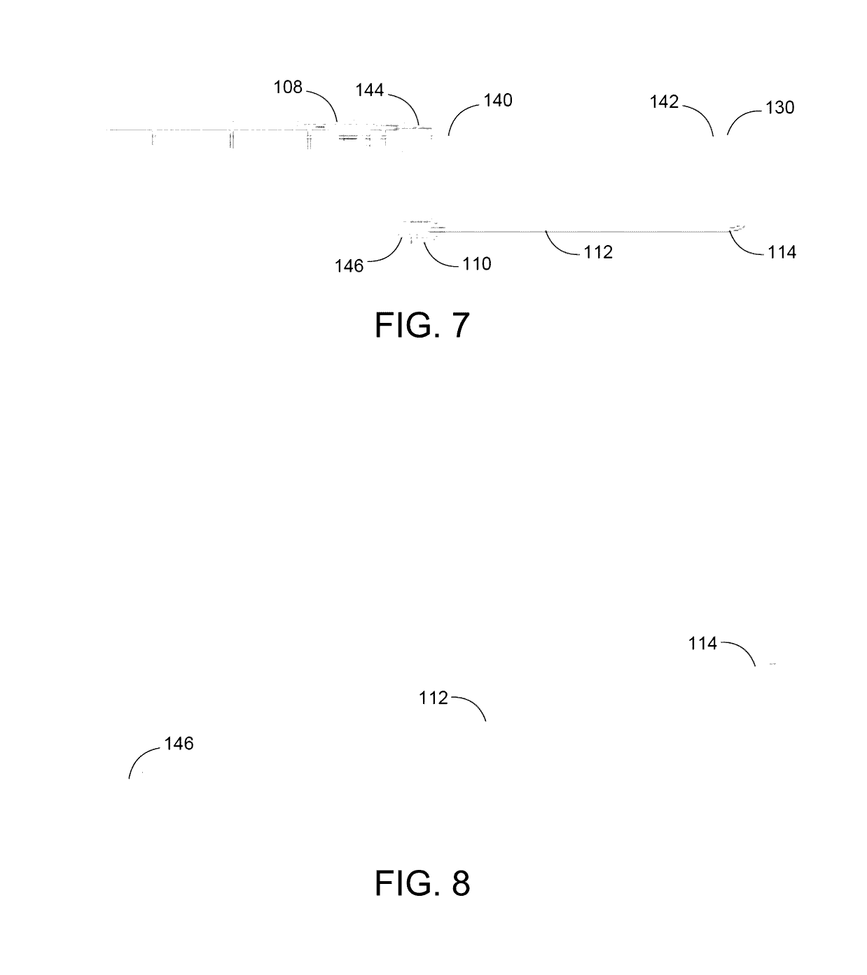

FIG. 7 illustrates a sectional side view of the disassembled cannula guided surgical tool system 100 of FIG. 5. FIG. 8 illustrates a more detailed view of the cannula assembly 104 of the surgical tool system 100 of FIG. 1.

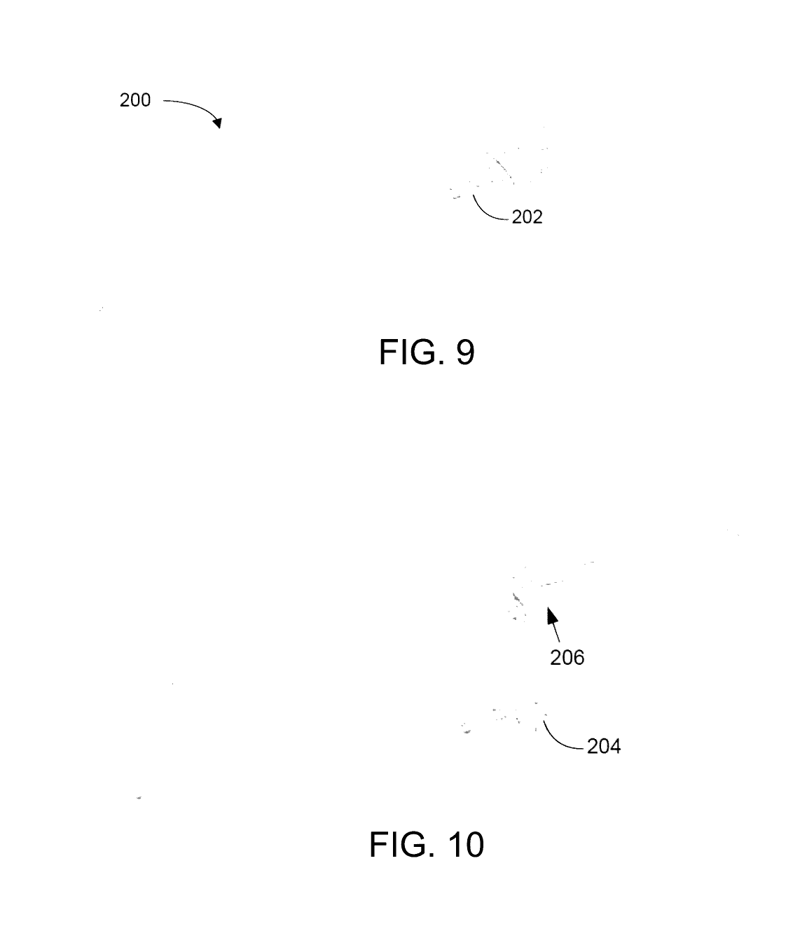

FIG. 9 illustrates another embodiment of a surgical tool system 200 with another type of connection mechanism. FIG. 10 illustrates another view of the surgical tool system 200 of FIG. 9. The illustrated system 200 includes a handle assembly and a cannula assembly that are connected together by a snap fit retention 202. The snap fit retention 202 includes spring loaded ears 204 that are retained in mating features 206 or holes built within the handle. The use of a snap fit retention 202 or other easily disconnected mating mechanism avoids the use of threaded parts which may be more expensive to manufacture and slightly more difficult to use in some situations.

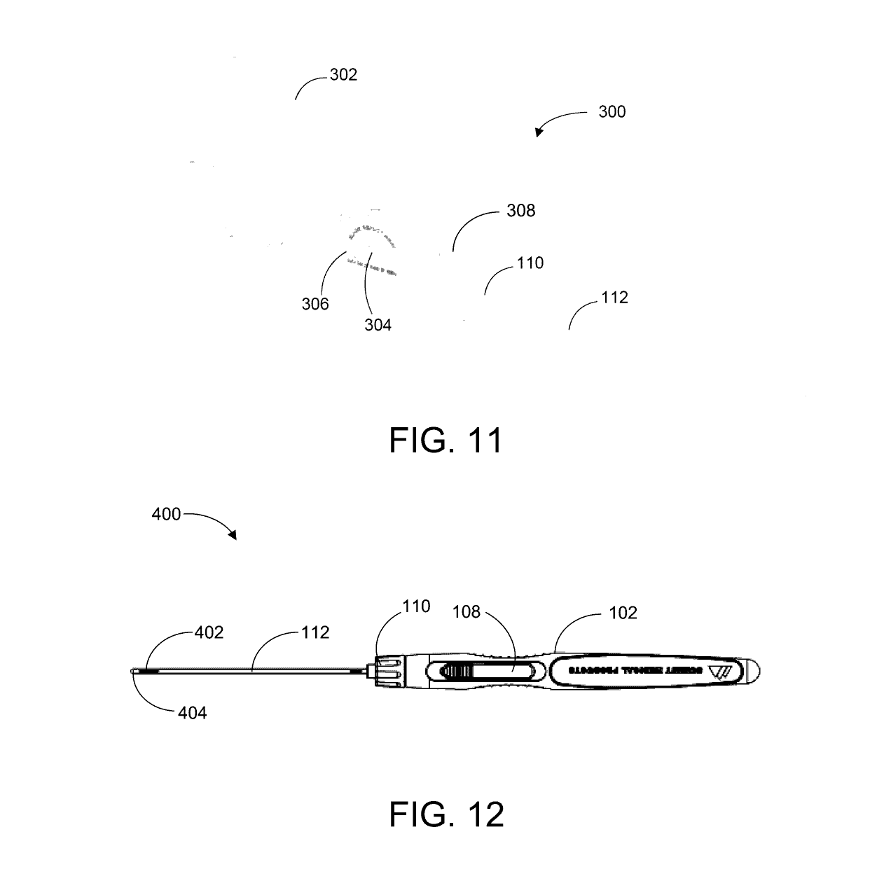

FIG. 11 illustrates another embodiment of a cannula guided surgical tool system 300 having a scissor handle assembly 302. The scissor handle assembly 302 may be more intuitive to use with certain types of surgical tools. For example, grasper or scissor tools may be controlled by a scissor type version of the handles versus a sliding actuator.

Additionally, some embodiments may include an additional luer port 308 (or other type of fluid port) that can permit the attachment of a fluid delivery system and flow of fluid to the treatment site. In this way, fluid may be delivered even while the blade or other surgical tool is still inserted in the cannula 112.

In some embodiments, the actuator 108 (such as the scissor handles 302) is coupled to a ratchet 304 or other counter that is used to count the deployments of the blade. By counting the deployments of the blade, the operator may ensure that only the desired quantity of deployments are allowed, because each deployment may have a degradation effect on the sharpness, accuracy, or other properties of the surgical tool. Once the deployment quantity has been achieved, a visual indicator 306 may be visible in a window aperture. Additionally, the actuator 108 or handles 302 may be locked mechanically to prevent further operations. In other embodiments, the counting, indicating, and locking mechanisms may be implemented using electronics or electromechanical features.

FIG. 12 illustrates another embodiment of a cannula guided surgical tool 400 having a tool port 402 on a sidewall of the cannula 112. The depicted tool 400 can be implemented with any type of cannula. Some embodiments may be particularly suitable for implementation with a straight cannula 112.

The Straight cannula 112 is fabricated from hypodermic tubing that is cut to a prescribed length. A small slot 402 is machined in the distal end of the cannula 112. A small tip 404 is machined and attached to the distal end of the cannula 112. The machined tip 404 closes the distal end of the cannula 114 and is secured using any method of precision attachment methods. The tip 404 may be attached by any means, including E-beam welding or another method of permanent attachment at the opening in the cannula 112. The E-beam welding process is a welding process that is done in robotically with a beam that is very small in diameter permitting a very precise controlled weld. The cannula 112 may be joined to the needle sleeve similar to other embodiments described herein.

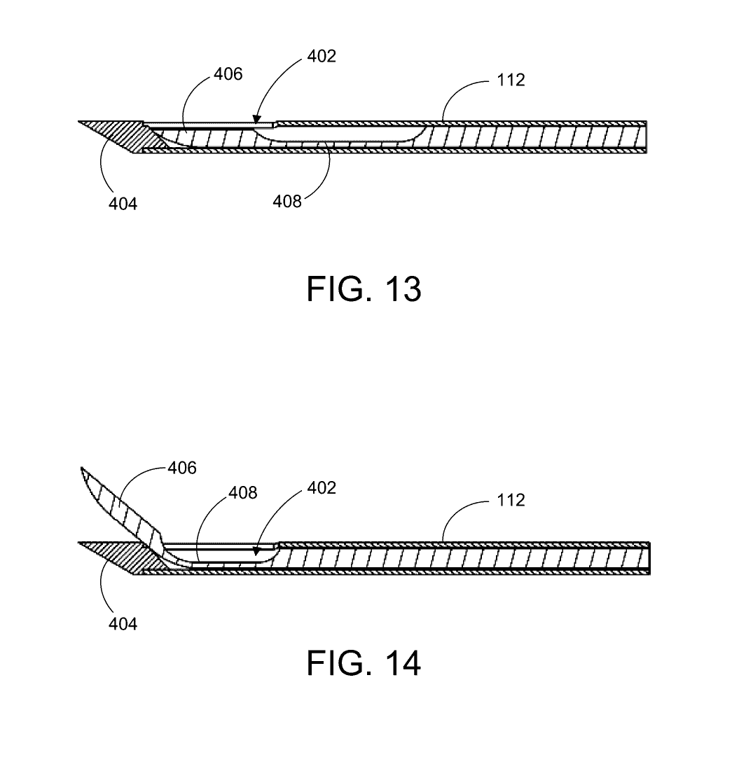

FIG. 13 illustrates a more detailed view of the ported cannula guided surgical tool 400 of FIG. 12. The distal end of the machined tip 404 is sharpened to a surgical sharpness and angle to form a surgical edge or point able to be used for tissue penetration. The proximal end of the machined tip 404 is machined at an angle and positioned within the cannula 112 to form an impact surface. The impact surface of the tip 404 may be angled relative to the opening slot 402 in the cannula 112. The positioning and geometry of the impact surface of the tip 404 allows a surgical tool 406 to impact or impinge on the impact surface and force the surgical tool to move laterally out of the slot 402 in the cannula 112. In some embodiments, the slot 402 is located on the top surface of the cannula 112 when the handle 102 and the needle are assembled to one another.

FIG. 14 illustrates a more detailed view of the ported cannula guided surgical tool 400 of FIG. 12 with the surgical tool 406 extending from the sidewall port 402. In one embodiment, the surgical tool is a blade with the same shape and configuration as the other embodiments described herein. When the actuator 108 is driven forward by the operator, the back surface of the blade impinges on the angled impact surface of the machined tip 404. This contact causes the blade to lift up and exit laterally or at an angle through the slot 402.

As described in other embodiments herein, the surgical tool includes a joint 408. In the illustrated embodiment, the joint is a flexible, thin section of wire or material. Since the blade is coupled to the flexible thin section, the blade will bend outward through the slot 402 at the thin section and cause the blade to continue up through the slot 402 at an angle that is controlled by the holding of the blade shaft within the cannula and the pressure exerted on the blade shaft against the proximal surface of the machined tip 404.

Further embodiments of the surgical tool systems may be scaled or scalable. This scalability may define the ability to make a family of tools with larger or smaller diameter cannulas. For example, a smaller diameter cannula supported tool may find use in cosmetic surgery fascia release procedures or neurology or percutaneous micro procedures where very small diameter tools are suitable or desirable to cause less superficial damage and encourage quicker healing. Visualization for such procedures and tools might follow the descriptions and coating methods described herein, including direct visualization.

Embodiments of the surgical tool systems described herein may be assembled and joined with the major assemblies secured together with the cap and packaged in a secure pouch to ensure protection of the blade or other surgical tool(s) during packaging, storage, shipment, and sterilizing. Removal of the pouch contents by the clinician will insure a sharp, sterile and ready to use tool for procedures as required by the clinician.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A surgical tool system comprising:
a cannula assembly comprising a cannula having a sidewall about a central axis; and
a handle assembly comprising a handle and a blade tool aligned with the central axis of the cannula, wherein the blade tool has a blade edge and a back edge, wherein the blade tool is longitudinally extendible relative to the handle;
wherein the blade tool is disposed within the cannula, and in a deployed mode the back edge of the blade tool impinges on an interior surface of the sidewall of the cannula to force the blade edge of the blade tool to protrude laterally from the central axis of the cannula to expose the blade edge of the blade tool beyond an outer diameter of the sidewall of the cannula.

2. The surgical tool system of claim 1, wherein the handle assembly further comprises an extension interface coupled to the blade tool, wherein the extension interface is configured to extend and retract the blade tool longitudinally through the cannula in response to movement of the extension interface.

3. The surgical tool system of claim 2, wherein the extension interface is configured to control how far the blade tool extends through the cannula and the blade edge protrudes from the central axis of the cannula.

4. The surgical tool system of claim 2, wherein the extension interface comprises a slider to slide longitudinally within the handle.

5. The surgical tool system of claim 4, further comprising a connector to attach the blade tool to the slider.

6. The surgical tool system of claim 2, wherein the handle further comprises a visual indicator with a series of extension marks, each extension mark indicative of an extension position of the blade tool out of the cannula at a corresponding position of the extension interface.

7. The surgical tool system of claim 1, wherein the cannula comprises a touhy cannula, and the interior surface of the touhy cannula is curved to force the blade tool, upon contact of the back edge of the blade tool with the interior surface of the touhy, to extend at a lateral angle away from the central axis of the cannula.

8. The surgical tool system of claim 1, wherein the cannula assembly further comprises a first coupling attached to the cannula,

9. The surgical tool system of claim 8, wherein the housing assembly further comprises a second coupled attached to the handle, the second coupling compatible to physically attach to the first coupling of the cannula assembly.

10. The surgical tool system of claim 9, wherein the first and second couplings engage through a twist-lock action.

11. The surgical tool system of claim 9, wherein the first and second couplings engage through a snap-lock action.

12. The surgical tool system of claim 1, wherein the blade tool further comprises an extension, a joint, and a blade with the blade edge and the back edge, wherein the joint facilitates bending in response to impingement of the back edge of the blade to angle the handle further comprises a visual indicator with a series of extension marks, each extension mark indicative of an extension position of the blade tool out of the cannula at a corresponding position of the extension interface.

13. The surgical tool system of claim 12, wherein the joint comprises a flexible web, wherein a length of the flexible web is at least 50% longer than a length of the blade.

14. The surgical tool system of claim 13, wherein the length of the flexible web is at least double the length of the blade.

15. The surgical tool system of claim 1, wherein the handle assembly further comprises a deployment counter to count a number of deployments of the blade tool and to provide an indication to a user when the number of deployments of the blade tool reaches a threshold number of deployments.

16. An surgical apparatus comprising:
a handle defining an interior cavity;
a needle cannula coupled to the handle, wherein a longitudinal axis of the needle cannula is aligned with the interior cavity of the handle;
a surgical tool extending from the interior cavity of the handle to a distal end of the needle cannula; and
an actuator within the handle, wherein the actuator comprises an extension interface coupled to the surgical tool to move the surgical tool longitudinally within the needle cannula and, in a deployed mode, extend the surgical tool out of the distal end of the needle cannula.

17. The surgical apparatus of claim 16, wherein the handle further comprises an extension controller to control the actuator, wherein the extension controller controls the extension of the surgical tool out of the distal end of the needle cannula within an established range of extension distances corresponding to movement positions of the actuator.

18. The surgical apparatus of claim 16, wherein the surgical tool comprises a surgical blade, and the actuator comprises a slider.

19. The surgical apparatus of claim 16, wherein the surgical tool comprises scissors, and the actuator comprises scissor handles.

20. The surgical apparatus of claim 16, wherein the surgical tool comprises a grasper, and the actuator comprises scissor handles.

21. The surgical apparatus of claim 16, wherein the needle cannula further comprises:
a tool port in a sidewall of the needle cannula; and
a needle tip disposed at a distal end of the needle cannula, wherein the needle tip comprises:
a surgical edge to penetrate tissue; and
an impact surface facing the surgical tool, wherein the surgical tool is configured to impinge on the impact surface of the needle tip to force at least a portion of the surgical tool to protrude laterally from the central axis of the needle cannula to expose the surgical tool beyond an outer diameter of the sidewall of the needle cannula.
